(19) United States
(12) Patent Application Publication
(10) Pub. No.: US 2011/0034954 A1
(43) Pub. Date: Feb. 10, 2011

(54) SUTURELESS REINFORCEMENT FOR AND METHOD OF TREATING A MYOCARDIAL INFARCTION

(75) Inventors: Jiahong Tan, Canyon Country, CA (US); Edward Sarnowski, Santa Monica, CA (US); Kevin L. Morgan, Simi Valley, CA (US)

Correspondence Address: PACESETTER, INC., 15900 VALLEY VIEW COURT, SYLMAR, CA, 91392-9221, US

(73) Assignee: PACESETTER, INC., Sylmar, CA (US)

(21) Appl. No.: 12/908,662

(22) Filed: Oct. 20, 2010

Related U.S. Application Data
(62) Division of application No. 11/735,192, filed on Apr. 13, 2007.

(51) Int. Cl. A61B 17/00

(52) U.S. Cl. 606/214; 606/213

(57) ABSTRACT

A method of treating a heart condition includes dispensing first and second sealant components to a myocardial infarction via an introducer extending through a percutaneous puncture in a subxiphoid region. The first and second sealant components combine to form a medical sealant material that bonds to the myocardial infarction to form a reinforcement for the myocardial infarction, wherein the reinforcement does not include a patch.

### CROSS REFERENCE TO RELATED APPLICATIONS

This application is a division of copending U.S. patent application Ser. No. 11/735,192, filed Apr. 13, 2007, titled “Sutureless Reinforcement For and Method Of Treating a Myocardial Infarction.”

### FIELD OF THE INVENTION

The invention relates generally to medical apparatus and methods and more specifically, to reinforcements for myocardial infarctions and methods of forming such reinforcements.

### BACKGROUND OF THE INVENTION

A myocardial infarction is the irreversible damage done to a segment of heart muscle by ischemia, where the myocardium is deprived of adequate oxygen and metabolite removal due to an interruption in blood supply. Ischemia is usually due to a sudden thrombotic occlusion of a coronary artery, commonly called a heart attack.

If the coronary artery becomes completely occluded and there is poor collateral blood flow to the affected area, an infarction can result. In some cases, the resulting infarction will be a transmural or full-wall thickness infarct in which much of the contractile function of the area is lost. Over a period of one to two months, the necrotic tissue heals, leaving a scar. The most extreme example of this is a ventricular aneurysm where all of the muscle fibers in the area are destroyed and replaced by fibrous scar tissue.

Even if the ventricular dysfunction as a result of the infarct is not immediately life threatening, common sequelae of a myocardial infarction in the left ventricle, whether the infarction is transmural or not, include mechanical cardiac disorders (e.g., heart remodeling such as ventricular remodeling or ventricular rupture) and electrical cardiac disorders. Such cardiac disorders lead to congestive heart failure where cardiac output falls below a level adequate to meet the metabolic needs of the body which, if uncompensated, leads to rapid death.

With respect to heart remodeling, whether the infarction is transmural or not, the damaged heart tissue may begin a long slow process of thinning and localized remodeling. Since the myocardial tissue is damaged and usually inadequately perfused, the damaged tissue contributes less to overall heart function compared to when the tissue was undamaged. The localized remodeling tends to place a greater demand on the rest of the heart to compensate for the reduced contractile capability of the damaged tissue. As a result, global heart function may be affected and, ultimately, the patient may experience global heart remodeling and descend into heart failure.

It is believed that preventing localized heart remodeling and the often-resulting globalized heart remodeling can prevent heart failure. Placing patches or substrates over the myocardial infarction is an emerging therapy believed to prevent localized heart remodeling and, as a result, global heart remodeling. The patches or substrates are used to reinforce the infarction and prevent the infarction from relaxing and expanding as time passes.

Such patches or substrates are implanted via highly invasive open chest procedures and are sutured or stapled to the infarct tissue or tissue adjacent to the infarct. Highly invasive open chest procedures significantly increase patient risk and recovery time. Also, infarct tissue or tissue adjacent to the infarct is often too fragile to receive sutures or staples.

There is a need in the art for a myocardial infarct reinforcement that does not require suturing/stapling. There is also a need for such a reinforcement that can be delivered via a minimally invasive procedure. There is also a need in the art for a method of treating a myocardial infarct that does not require suturing/stapling. There is also a need in the art for such a method that is minimally invasive.

### BRIEF SUMMARY OF THE INVENTION

Briefly, and in general terms, the invention relates to methods of treating myocardial infarction. One such method includes assembling a myocardial infarction reinforcement at a location of a myocardial infarction in a patient.

Another method of treating myocardial infarction includes impregnating a patch with a first sealant component; delivering the patch to a myocardial infarction via an introducer extending through a percutaneous puncture in a subxiphoid region of a patient; and delivering a second sealant component to the myocardial infarction via the introducer extending through the percutaneous puncture in the subxiphoid region, wherein the first and second components combine to form a medical sealant material that bonds the patch to the myocardial infarction, thereby forming a reinforcement for the myocardial infarction.

Yet another method of treating myocardial infarction includes dispensing first and second sealant components to a myocardial infarction via an introducer extending through a percutaneous puncture in a subxiphoid region, the first and second sealant components combine to form a medical sealant material that bonds to the myocardial infarction to form a reinforcement for the myocardial infarction, wherein the reinforcement does not include a patch.

The invention also relates to myocardial infarction reinforcements. One such reinforcement includes a patch and medical sealant material. The patch includes a sheet having pores/voids. The patch extends over a myocardial infarction. The medical sealant material impregnates the pores/voids and bonds the patch to the myocardial infarction.

Another reinforcement includes a medical sealant material extending over and bonding to a myocardial infarction to form the reinforcement.

While multiple embodiments are disclosed, still other embodiments of the present invention will become apparent to those skilled in the art from the following detailed description, which shows and describes illustrative embodiments of the invention. As will be realized, the invention is capable of modifications in various aspects, all without departing from the spirit and scope of the present invention. Accordingly, the drawings and detailed description are to be regarded as illustrative in nature and not restrictive.

### BRIEF DESCRIPTION OF THE DRAWINGS

FIG. 1 is a plan view of a patch used to form a myocardial infarction reinforcement.

FIG. 2 is a cross section of the patch as taken along section line 2-2 in FIG. 1.

FIG. 3 is a diagrammatic plan view of a patient with the chest open to reveal the patch being located intrapericardially via an introducer extending through a percutaneous puncture in the subxiphoid region.

FIGS. 4-7 are diagrammatic views of the patch at various stages of its deployment via the introducer.

FIG. 8 is a cross section view of the patch as taken along section line 2-2 in FIG. 2, except the patch is deployed in the intrapericardial space and adhered to the epicardial surface via the sealant to form the reinforcement of the myocardial infarction.

FIG. 9 is a perspective view of a patch in a non-deployed state being passed through a lumen of an introducer.

FIG. 10 is the same view depicted in FIG. 9, except the patch has biased into the expanded or deployed state and is being maneuvered into place over a myocardial infarction via a stylet distal end.

FIG. 11 is the same view depicted in FIGS. 4-7, except sealant is being applied prior to the deployment of the patch to the myocardial infarction 4.

FIG. 12 is the same view as FIGS. 4-7, except the sealant is being applied subsequent to the deployment of the patch to the myocardial infarction.

FIGS. 13 and 14 are views similar to FIGS. 11 and 12, except illustrating a creation of a myocardial infarction reinforcement that does not employ a patch.

### DETAILED DESCRIPTION

The present application describes suture-free/staple-free reinforcements for, and methods of, treating a myocardial infarction that is leading to, or already has lead to, ventricular remodel or rupture. The reinforcements can be delivered and the treatment method can be performed by open chest procedures or, advantageously, by minimally invasive procedures such as accessing the intrapericardial space surrounding the heart via a puncture in the pericardial sac and a percutaneous puncture in the subxiphoid region of a patient.

In one embodiment, the reinforcement is one or more layers of medical adhesive and/or sealant material (generically referred to in the rest of this Detailed Description as medical sealant material) directly applied to the epicardial surface of a myocardial infarction. In other embodiments, the reinforcement is a patch adhered to the epicardial surface of a myocardial infarction via the sealant material. The sealant material can impregnate the patch and/or be applied prior to, during, and/or subsequent to the patch being located over the myocardial infarction. Regardless of whether the reinforcement is formed via a patch/sealant combination or solely with sealant, the reinforcement adheres to and reinforces the myocardial infarction without the use of sutures or staples. Also, for some embodiments, the reinforcement can be said to be formed, assembled or created in the patient at the location of the myocardial infarction.

With reference to FIGS. 1-3, in one embodiment, the patch 22 includes a frame 26 and a mesh or porous sheet 24 extending between sides or sections of the frame 26. In other embodiments, the patch 22 will not include a frame 26, but will simply be the sheet 24.

The sheet 24 may be attached to one side of the frame 26. Alternatively, the sheet 24 extends about the frame 26 (e.g., the frame 26 is received in a seam of the sheet 24). The size of the sheet 24 may be generally the same as the size of the frame 26 or it may exceed the size of the frame 26 such that the edge of the sheet 24 extends past the frame 26.

Sizes of the patch 22 in their respective deployed, flattened states vary depending on the size of the heart 7 and the size of the infarcted area 4. In one configuration, the patch 22 in its deployed, flattened state is at least approximately four millimeters by at least approximately seven millimeters. In another configuration, the patch 22 in its deployed, flattened state has a diameter of between approximately five millimeters and approximately eight millimeters. With respect to thickness, in one configuration, the patch 22 it its deployed, flattened state has a thickness of between approximately 0.2 millimeters and approximately 0.5 millimeters, while in another configuration it has a thickness of between approximately 0.1 millimeters and approximately 2 millimeters.

The patch 22 in its deployed, flattened state is preferably at least approximately the same size as the area of the myocardial infarction. In one embodiment, the patch 22 in its deployed, flattened state has a size that exceeds the size of the area of the myocardial infarction by approximately zero percent to approximately 20 percent or more.

The sheet 24 is fixed to the frame 26 via molding, gluing, suturing, or welding (e.g., sonic, chemical, heat, laser, etc.). The frame 26 may be formed of Nitinol, polytetrafluoroethylene (“PTFE”), silicone rubber, polyurethane, etc. The sheet 24 may be formed of a mesh or porous biocompatible natural material (e.g., a sheet of pericardium, etc.) or a mesh or porous biocompatible synthetic material (e.g., DACRON®, polyester, polyurethane, absorbable polymers, polypropylene, PTFE, e-PTFE, hydro-gel, Titanium mesh, stainless steel mesh, and/or etc.). In one embodiment, the sheet 24 is a mesh that is fabric knitted from very thin yarn with pores, having 5-20 Wales count/cm, 10-30 course count/cm.

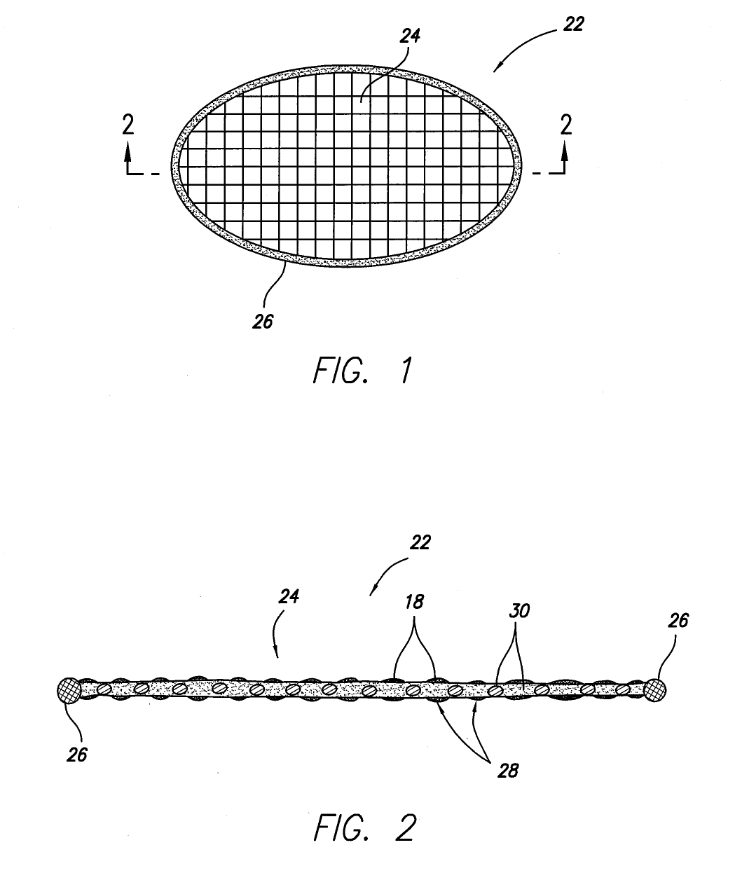

Regardless of the material selected for the sheet 24, in one embodiment, the material selected for the sheet 24 is such that the sealant 18 can penetrate the material of the sheet 24 to assist in bonding the sheet 24 to a myocardial surface 20. For example, as indicated in FIG. 2, in one embodiment, the sheet 24 has micro pores/voids 28 defined between threads/structures 30 forming the porous or mesh material of the sheet 24. Thus, the sealant material 18 can impregnate the micro pores/voids 28 in the sheet 24.

In one embodiment, the sheet 24 is loaded with sealant 18 prior to being delivered through the introducer 32. The sheet 24 is loaded with the sealant 18 according to a defined ratio. In other embodiments, the sealant 18 is applied to the sheet 24 subsequent to the patch 22 being delivered to the pericardial space 6. In either case, the sealant 18 impregnates the pores/voids 28 and assists in adhering the patch 22 to the epicardial surface 20 to form the reinforcement 2 over the myocardial infarction 4.

In one embodiment, the sheet 24 has different surface designs or conditions on its flat faces. For example, in one embodiment, the sheet-face facing the pericardium will be smooth (e.g., a non-porous structure) and the sheet-face facing the epicardial surface is porous. The smooth sheet-face facing the pericardium will mitigate interaction of the pericardium with the sheet 24 to decrease tissue ingrowth from the pericardium. The porous sheet-face facing the epicardial surface will facilitate the sealant impregnating the sheet to bond the patch 22 to the epicardial surface.

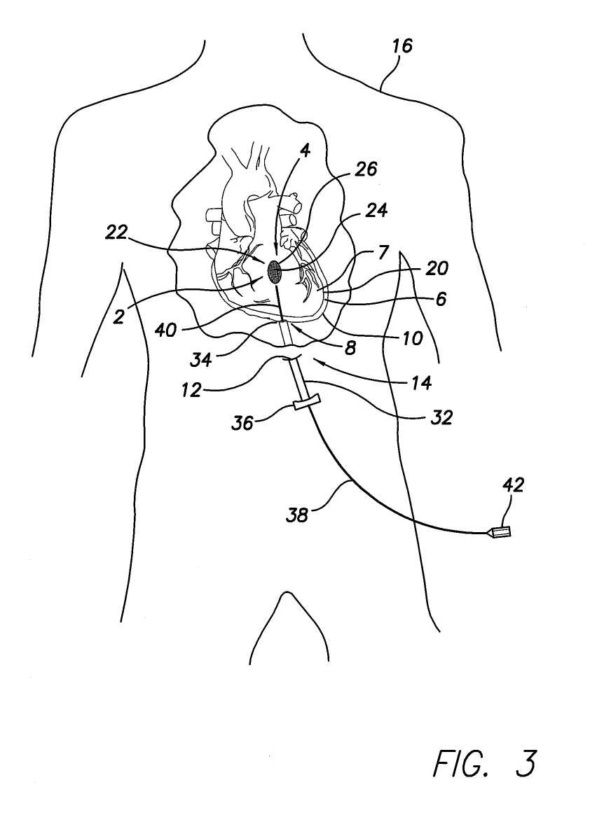

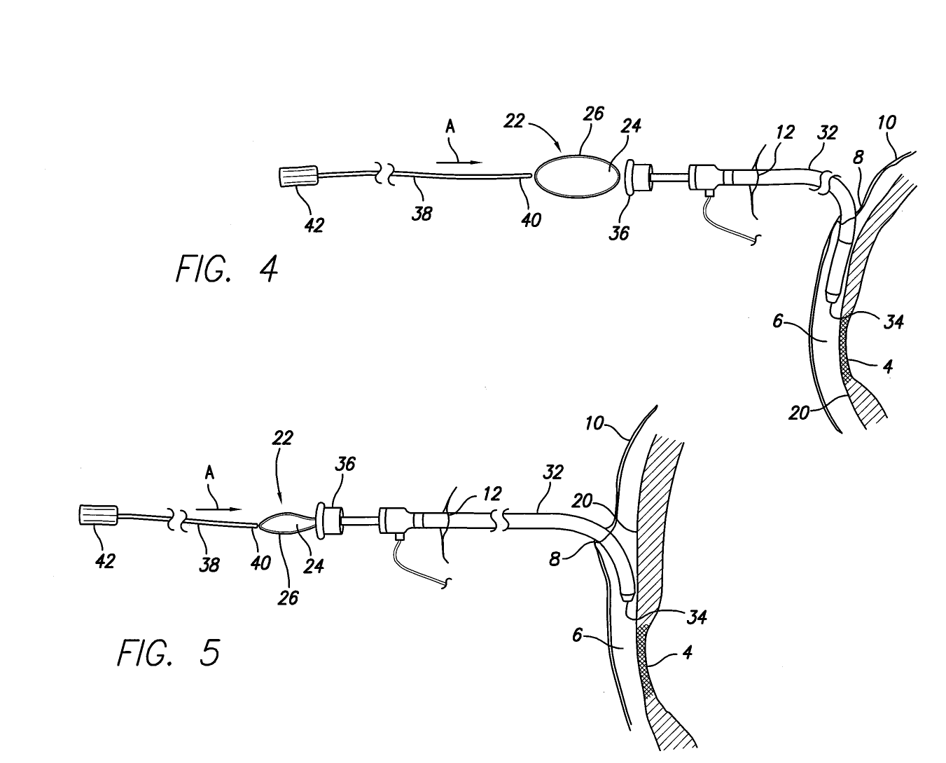

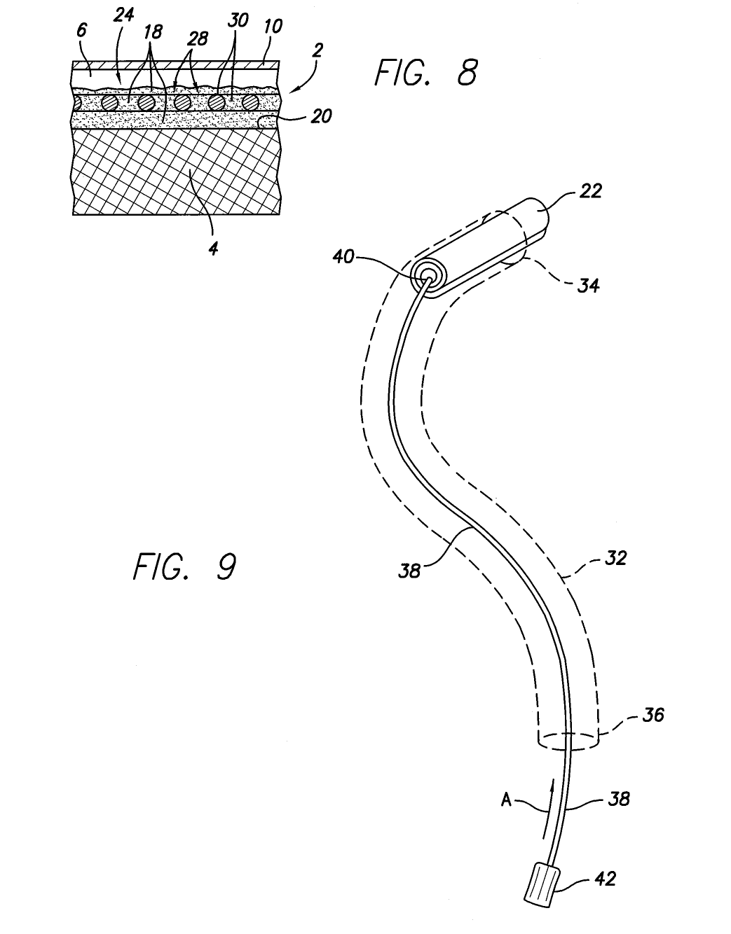

For a detailed discussion of a method of deploying the patch 22 to form a reinforcement 2 over a myocardial infarction 4, reference is made to FIGS. 3-8. FIG. 3 is a diagrammatic plan view of a patient 16 with the chest open to reveal the patch 22 being located intrapericardially via an introducer 32 extending through a percutaneous puncture 12 in the subxiphoid regionl4. FIGS. 4-7 are diagrammatic views of the patch 22 at various stages of its deployment via the introducer 32. FIG. 8 is a cross section view of the patch 22 as taken along section line 2-2 in FIG. 2, except the patch 22 is deployed in the intrapericardial space 6 and adhered to the epicardial surface 20 via a sealant 18 to form the reinforcement 2 of the myocardial infarction 4.

The myocardium infarction 4 may be located via an imaging system (e.g., via an echo-graphic system). As can be understood from FIG. 3, the pericardium 10 surrounding the heart 7 is accessed via a percutanous puncture 12 in the subxiphoid area 14 of the patient 16 and a puncture 8 in the pericardial sac 10. The punctures 12, 8 may be made via a Touhy needle viewed under fluoroscopy. A guide wire is placed through the central lumen of the Touhy needle into the pericardial space 6. The Touhy needle is withdrawn and an introducer 32 is placed over the guidewire (not shown) into the pericardial space 6 and the distal end 34 of the introducer 32 is positioned near the myocardium infarction 4.

The guidewire is removed from within the introducer 32 and, as indicated in FIG. 4, the patch 22 is positioned near a proximal end 36 of the introducer 32. The introducer 32 extends into the pericardial space 6 via the percutanous puncturel2 in the subxiphoid area 14 and the pericardial sac puncture 8 such that the distal end 34 of the introducer 32 is positioned near the myocardial infarction 4 of the heart 7 of the patient 16.

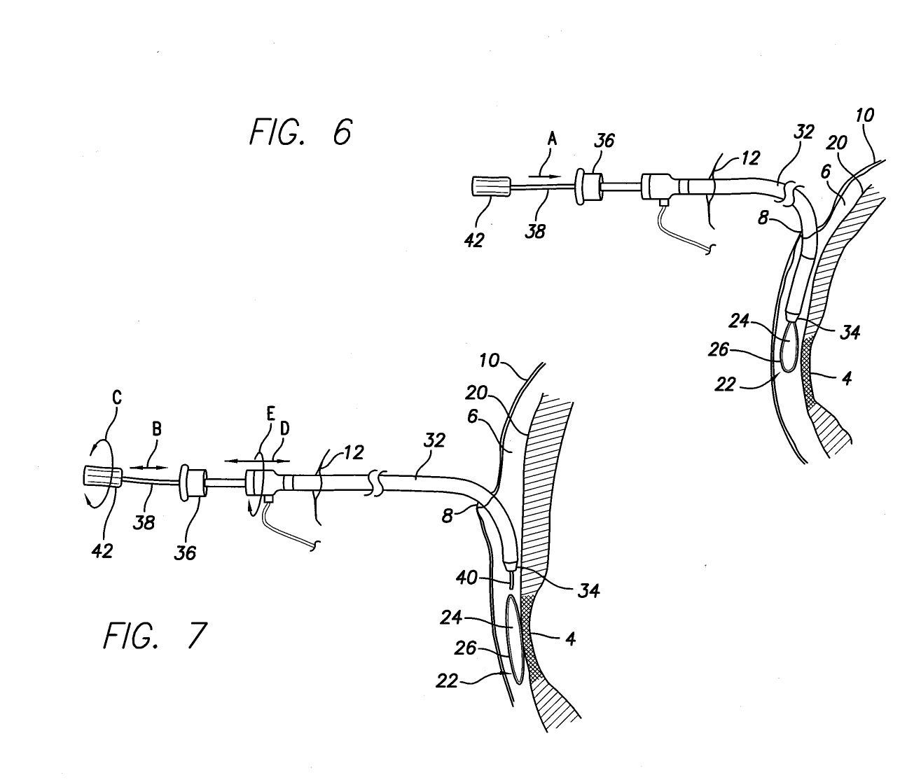

As depicted in FIG. 5, the patch 22 is collapsed from a deployed or expanded condition (illustrated in FIGS. 1 and 4) to a collapsed or non-deployed condition so as to allow the patch 22 to be inserted in the lumen of the introducer sheath 32. As can be understood from FIGS. 5 and 6, a pushing member (e.g., a stylet) 38 is displaced distally (as indicated by arrow A) such that a distal end 40 of the stylet 38 contacts and distally moves the collapsed patch 22 through the lumen of the introducer 32 to eventually protrude from the distal end 34 of the introducer 32. As indicated in FIG. 7, once the patch 22 fully emerges from the lumen of the introducer distal end 34, the patch 22, which is biased to expand or deploy, expands to the deployed or expanded condition best illustrated in FIGS. 1 and 4. As indicated by arrows B, C, D and E, the distal ends 40, 34 of the stylet 38 and/or introducer 32 are rotated and distally and/or proximally displaced via manipulation of their respective proximal ends 42, 36 to manipulate the patch 22 into position over the myocardial infarction 4.

As can be understood from FIGS. 7 and 8, the fully expanded or deployed patch 22 is placed over the epicardial surface 20 of the infarct 4 such that the sheet 24 extends across the epicardial surface 20 in a plane generally parallel to the epicardial surface 20. As can be understood from FIGS. 4-6, in one embodiment, the patch 22 is configured to collapse in a direction transverse to the direction of travel through the introducer 32, thereby allowing the patch 22 to pass through the introducer lumen. Upon exiting the distal end of the introducer lumen, the patch 22 expands transversely back to the expanded state depicted in FIGS. 1 and 4.

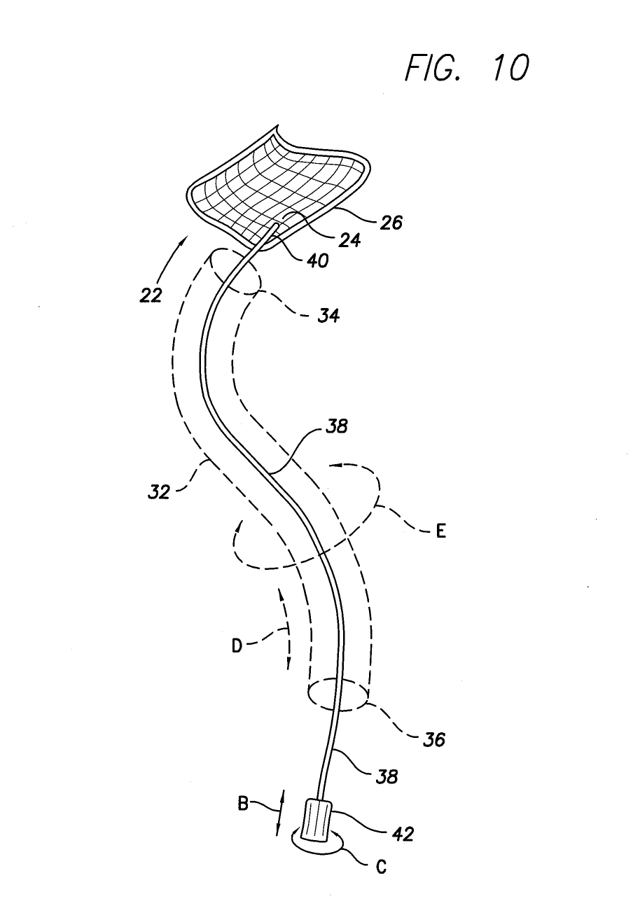

In other embodiments, the patch 22 expands in other manners between the non-deployed and deployed states as illustrated in FIGS. 9 and 10. FIG.9 is a perspective view of a patch 22 in a non-deployed state being passed through a lumen of an introducer 32. FIG. 10 is the same view depicted in FIG. 10, except the patch 22 has biased into the expanded or deployed state and is being maneuvered into place over a myocardial infarction via a stylet distal end 40.

As shown in FIG. 9, the patch 22 is rolled up and passed through the introducer lumen as a roll. As indicated by arrow A, the stylet 38 is used to distally push the rolled patch 22 through the introducer lumen. The wall of the introducer lumen maintains the patch 22 in the rolled state until the roll exits the introducer distal end 34 to bias into the expanded or deployed state depicted in FIG. 10. As indicated by arrows B, C, D, and E, the stylet proximal end 42 and/or introducer proximal end 36 are rotated and/or longitudinally displaced to manipulate their respective distal ends 40, 34 to maneuver the patch 22 into position over the myocardial infarction 4.

Regardless of whether the patch deployment configuration is as depicted in FIGS. 4-7 or FIGS. 9-10, in one embodiment, the patch 22 is loaded with sealant 18 prior to the patch 22 being deployed through the introducer 32. The sealant 18 impregnating the sheet 24 of the patch 22 adheres the underside of the patch 22 to the epicardial surface 20, thereby forming the reinforcement 2 over the myocardial infarction 4. The sealant 18 is preferably present in the patch 22 in sufficient amounts to adequately bond the patch 22 to the epicardial surface 20 without the application of any other amounts of sealant 18. In one such embodiment, the medical sealant material 18 is a fibrin sealant, a gelatin-resorcinol-formaldehyde glue, a hydrogel sealant, an alginate, a cyanoacrylate ester, or etc.

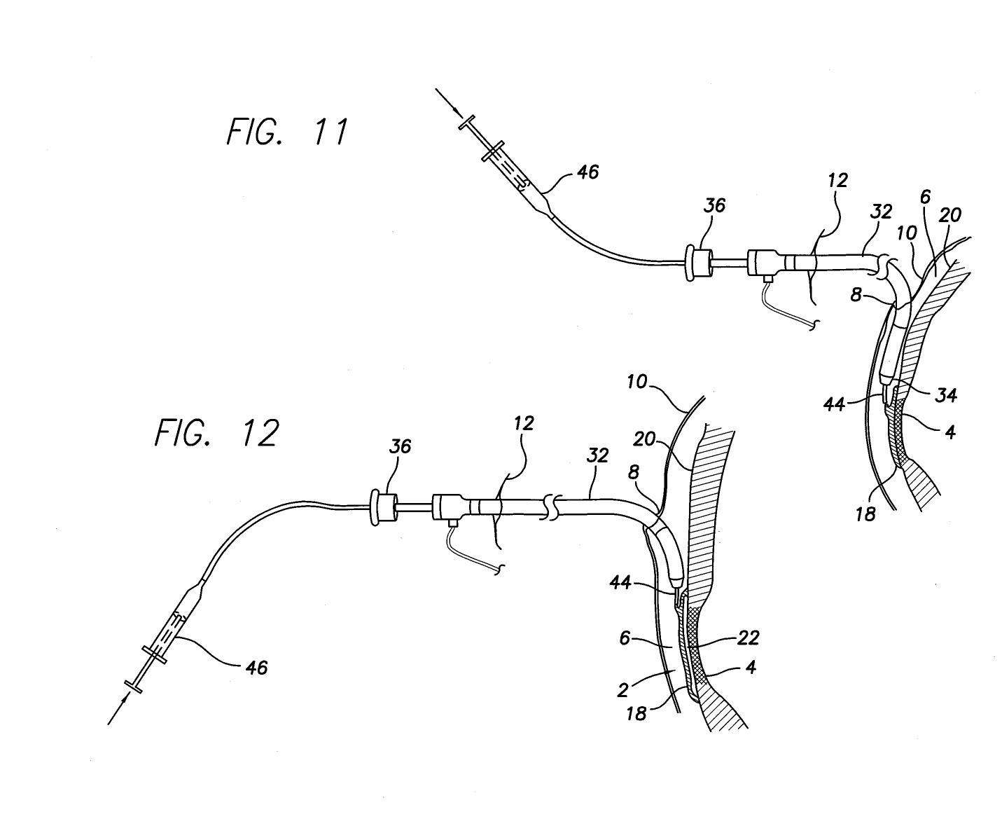

In some embodiments, sealant 18 is applied prior to and/or subsequent to the deployment of the patch 22 over the myocardial infarction 4. Such embodiments are illustrated in FIGS. 11 and 12. FIG. 11 is the same view depicted in FIGS. 4-7, except sealant 18 is being applied prior to the deployment of the patch 22 to the myocardial infarction 4. FIG. 12 is the same view as FIGS. 4-7, except the sealant 18 is being applied subsequent to the deployment of the patch 22 to the myocardial infarction 4.

As shown in FIG. 11, a distal end 44 of a sealant-dispensing syringe 46 is passed through the introducer lumen to protrude from the introducer distal end 34. Sealant 18 is applied to the epicardial surface 20 at the myocardial infarction 4 via the syringe distal end 44. A patch 22 (not shown) is then deployed over the myocardial infarction 4 and sealant 18 per one of the methods depicted in FIGS. 4-10.

In one embodiment, the patch 22 is loaded with sealant. The sealant 18 applied to the epicardial surface 20 prior to the patch deployment and the sealant carried by the patch 22 combine to provide sufficient adherence of the patch 22 to the epicardial surface 20 over the myocardial infarction 4 to form the reinforcement 2.

In another embodiment, the patch 22 is not loaded with sealant. The sealant 18 applied to the epicardial surface 20 prior to the patch deployment is by itself sufficient to provide sufficient adherence of the patch 22 to the epicardial surface 20 over the myocardial infarction 4 to form the reinforcement 2.

In alternative versions of either of the two immediately preceding embodiments, once a sealant 18 is deployed as depicted in FIG. 11 and the patch 22 is deployed as illustrated in FIGS. 4-10, sealant 18 is applied over the outer surface of the patch 22 via the distal end 44 of a sealant dispensing syringe 46. The sealants 18 applied prior and subsequent to the deployment of the patch 22 and the sealant 18 (if any) loaded in the patch 22 combine to sufficiently adhere the patch 22 to the epicardial surface 20 over the myocardial infarction 4 to form the reinforcement 2.

With reference to FIG. 12, in one embodiment, the patch 22 is deployed as illustrated in FIGS. 4-10. However, unlike the immediately preceding embodiments discussed with reference to FIG. 11, no sealant 18 is applied to the epicardial surface 20 prior to the deployment of the patch 22. Once the patch 22 is deployed, a sealant 18 is applied over the outer surface of the patch 22. The sealant 18 applied subsequent to the patch deployment and the sealant (if any) loaded in the patch 22 combine to sufficiently adhere the patch 22 to the epicardial surface 20 over the myocardial infarction 4 to form the reinforcement 2.

In one embodiment, the sealant (if any) loaded in the patch 22 and each application of sealant 18, whether applied prior to or subsequent to the patch deployment, is the same type of sealant, although the sealant may be applied in one or more sealant components, as discussed below.

In one embodiment, the medical sealant material 18 is a fibrin sealant such as CROSSEAL™ as manufactured by OMRIX Biopharmaceuticals, Ltd. of Belgium or TISSEEL™ as manufactured by Fuer Haemoderivate G.M.B.H. of Vienna, Austria. In one embodiment as discussed below, the fibrin sealant 18 is formed from two or more different medical sealant components. For example, a first applied sealant component is a solution containing fibrinogen and Factor XIII, and the second applied sealant component is a solution containing thrombin and CaCl₂. On mixing the two sealant components using a device such as a twin syringe with a mixing nozzle, the sealant components react. The reaction is similar to the final stages of blood clotting and results in polymerization of the fibrinogen to fibrin monomers and a white fibrin clot is initiated under the action of thrombin and CaCl₂. The fibrin sealant material 18 is hemostatic, biodegradable, is associated with excellent tissue tolerance, readily adheres to connective tissue and promotes wound healing.

In another embodiment, the medical sealant material 18 is a cyanoacrylate ester, which is a fluid monomer that polymerizes rapidly in the presence of weak bases such as water or NH₂ groups. The cyanoacrylates spread rapidly on the surface of a myocardial infarction 4 and polymerize rapidly in the presence of blood. These materials achieve rapid hemostasis as well as a strong bond to tissue.

In another embodiment, the medical sealant material 18 is gelatin-resorcinol-formaldehyde glue. The glue is fabricated by warming a 3:1 mixture of gelatin and resorcinol and adding an 18% formaldehyde solution. Cross-linking of the gelatin and resorcinol by the formaldehyde takes place in about 30 seconds to provide rapid hemostasis and a strong tissue bond.

In yet another embodiment, the medical sealant material 18 is a polyethylene glycol (“PEG”) hydrogel. A PEG hydrogel, as manufactured by Focal, Inc. of Lexington, Mass., is formed by in situ macromers followed by photopolymerization to highly cross-linked structures. The resulting medical sealant material 18 provides rapid hemostasis and a strong tissue bond.

In still another embodiment, the medical sealant material 18 is an alginate. The resulting medical sealant material 18 provides rapid hemostasis and a strong tissue bond.

As mentioned above, in some embodiments, the medical sealant material 18 is formed via two or more medical sealant components reacted together. The medical sealant components 18 may be applied to the myocardial infarction 4 at the same instance or in separate instances. For example, in one embodiment, a first medical sealant component is a low molecular weight monomer or oligomer, and a second medical sealant component is a catalyst or another reactant. When the catalyst is combined with the monomer to create the reinforcement 2, the catalyst causes the monomer to rapidly polymerize. Similarly, when the reactant is combined with the oligomer to create the reinforcement 2, the reactant causes the oligomer to rapidly polymerize.

As can be understood from FIG. 11, in one embodiment of a two-component medical sealant material 18, a first sealant component (e.g., a low molecular weight monomer or an oligomer) is first provided at the myocardial infarction 4. The patch 22 (not shown), which is loaded with a second sealant component (e.g., a catalyst or reactant), is then applied to the first sealant component. The first and second sealant components react to rapidly polymerize the sealant component, achieving rapid hemostasis and a strong adhesion of the patch 22 and medical sealant to the myocardial infarction 4, thereby forming the reinforcement 2.

In another embodiment of a two component medical sealant material 18, a first sealant component (e.g., a low molecular weight monomer or an oligomer) is first provided at the myocardial infarction 4. The patch 22, which may or may not be loaded with one of the sealant components) is then applied to the myocardial infarction 4. The second sealant component (e.g., a catalyst or reactant) is then applied to the patch 22 and first sealant component. The first and second sealant components react to rapidly polymerize the sealant component, achieving rapid hemostasis and a strong adhesion of the patch 22 and medical sealant 18 to the myocardial infarction 4, thereby forming the reinforcement 2.

As can be understood from FIG. 12, in yet another embodiment of a two component medical sealant material, a patch 22 loaded with a first sealant component (e.g., a low molecular weight monomer or an oligomer) is first provided at the myocardial infarction 4. The second sealant component (e.g., a catalyst or reactant) is then applied to the patch 22 and the first sealant component carried by the patch 22. The first and second sealant components react to rapidly polymerize the sealant component, achieving rapid hemostasis and a strong adhesion of the patch 22 and medical sealant 18 to the myocardial infarction 4, thereby forming the reinforcement 2.

As best understood with reference to FIG. 11, in one embodiment, the sealant 18 provided at the myocardial infarction 4 prior to the deployment of the patch 22 is formed of a first sealant component (e.g., a low molecular weight monomer or an oligomer) and a second sealant component (e.g., a catalyst or reactant). The first and second sealant components are applied via a twin syringe (not shown) immediately prior to the deployment of the patch 22. The first and second sealant components react to achieve rapid hemostasis and a strong adhesion of the patch (not shown) and medical sealant 18 to the myocardial infarction 4, thereby forming the reinforcement 2.

As best understood with reference to FIG. 12, in one embodiment, the patch 22 is deployed at the myocardial infarction 4. The sealant 18 provided over the patch at the myocardial infarction 4 subsequent to the deployment of the patch 22 is formed of a first sealant component (e.g., a low molecular weight monomer or an oligomer) and a second sealant component (e.g., a catalyst or reactant). The first and second sealant components are applied via a twin syringe (not shown) subsequent to the deployment of the patch 22. The first and second sealant components react to achieve rapid hemostasis and a strong adhesion of the patch 22 and medical sealant 18 to the myocardial infarction 4, thereby forming the reinforcement 2.

In a first version of any one of the immediately preceding embodiments, the first sealant component is a low molecular weight monomer or an oligomer, and the second sealant component is a catalyst or reactant. In second version of any one of the immediately preceding embodiments, the first sealant component is a solution containing fibrinogen and Factor XIII, and the second sealant component is a solution containing thrombin and CaCl₂. In a third version of any one of the immediately preceding embodiments, the first sealant component is a solution containing a 3:1 mixture of gelatin and resorcinol, and the second sealant component is a solution of 18% formaldehyde.

While each of the immediately preceding embodiments is discussed in the context of two sealant components reacting to form a sealant material 18, in other embodiments, there may be more than two sealant components reacting to form a sealant material. Also, the sealant components may be applied or provided in combinations and sequences other than those examples provided above. Such other combinations and sequences should be considered within the scope of this Detailed Description.

For any of the preceding and following embodiments employing a sealant material 18 formed of multiple sealant components, the fixing time of the patch 22 to the epicardial surface can be controlled by the mixing ratio of the sealant components. In other words, the time for the sealant material to cure and affix or form the patch over the infarction can be increased or decreased depending on the mixing ratio of a first sealant component relative to the other sealant component(s) forming the sealant material.

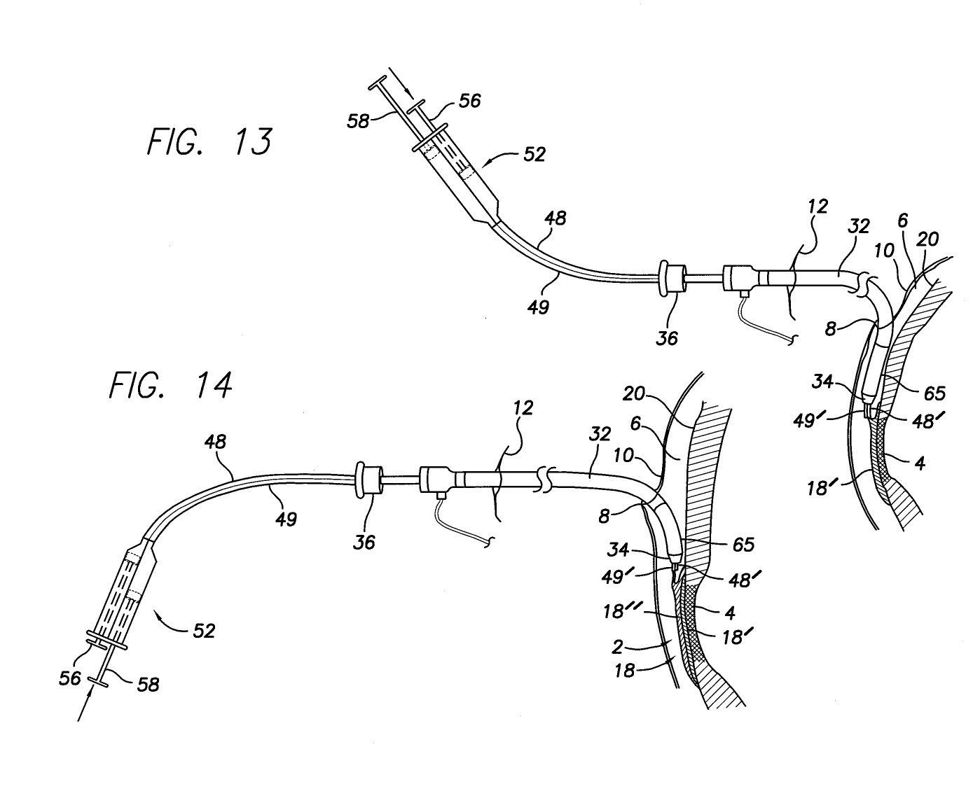

While the preceding embodiments employ a patch 22 in the formation of the reinforcement 2 for the myocardial infarction, in other embodiments the reinforcement 2 will be formed without a patch 22. For example, as depicted in FIGS. 13 and 14, which are views similar to FIGS. 11 and 12, first and second tubes 48, 49 leading from twin separate nozzles of a twin chamber syringe 52 are fed through the introducer 32 such that the tube distal ends 48′, 49′ protrude from the introducer 32 near the myocardial infarction 4. As shown in FIG. 13, a first plunger 56 is distally displaced within the syringe 52 to dispense through the first tube 48 any one of the above-discussed first sealant components 18′ (e.g., a solution of fibrinogen and XIII) onto the epicardial surface 20 of the myocardial infarction 4. As illustrated in FIG. 14, a second plunger 58 is distally displaced within the syringe 52 to dispense through the second tube 49 onto the first sealant component 18′ any one of the above-discussed second sealant components 18″ (e.g., a solution of thrombin and CaCl₂) compatible with the applied first sealant component 18′. The first and second sealant components 18′, 18″ react to rapidly polymerize into the sealant 18, which strongly adheres to the heart tissue and achieves rapid hemostasis. The sealant 18 dispensed on the myocardial infarction 4 forms the reinforcement 2, which does not employ a patch 22.

In another embodiment, the twin syringe 52 and the first and second sealant components 18′, 18″ are the same as discussed with respect to FIGS. 13 and 14, except the syringe 52 has a single mixing nozzle and a single dispensing tube extending from the mixing nozzle. The single dispensing tube extends through the introducer 32 to the myocardial infarction 4. Both plungers 56, 58 are distally displaced together within the syringe 52, causing the first and second sealant components 18′, 18″ to mix in the mixing nozzle and be dispensed as a mixed sealant material 18 onto the myocardial infarction 4. The mixed sealant material 18 forms the reinforcement, which does not employ a patch 22.

Regardless of whether the reinforcement 2 is formed of a combination of patch 22 and medical sealant material 18 or solely of a medical sealant 18, the reinforcement 2 is advantageous for a number of reasons. First, although the reinforcement 2 can be applied to a myocardial infarction 4 via an open chest procedure, it is can be advantageously performed via a minimally invasive procedure such as a percutaneous puncture 12 in the subxiphoid region 14 of a patient 16. Second, the reinforcement 2 affixes to the epicardial surface 20 of or near a myocardial infarction 4 via the adhesive qualities of the medical adhesive or sealant 18 used to form the reinforcement, thereby avoiding the need for sutures/staples and the issues associated therewith. Third, the reinforcement 2 mechanically constrains the heart tissue at the myocardial infarction 4, thereby preventing, or at least reducing, dilation of the left ventricle by supporting and thickening the area that has been thinned due to the myocardial infarction scar. Preventing the dilation of the left ventricle may prevent, or at least mitigate, left ventricle remodeling that can eventually lead to heart failure in post myocardial infarction patients.

Although the present invention has been described with reference to preferred embodiments, persons skilled in the art will recognize that changes may be made in form and detail without departing from the spirit and scope of the invention.

What is claimed is:

1. A method of treating a heart condition, the method comprising assembling a myocardial infarction reinforcement at a location of a myocardial infarction in a patient, wherein the reinforcement does not include a patch.

2. The method of claim 1, wherein the reinforcement is assembled by applying a medical sealant material to the myocardial infarction.

3. The method of claim 2, wherein the medical sealant is formed from a first sealant component combined with a second sealant component.

4. The method of claim 3, wherein the combining of the components occurs at the location of the myocardial infarction.

5. The method of claim 3, wherein the combining of the components occurs outside the patient and the combined components are then delivered to the myocardial infarction to assemble the reinforcement.

6. The method of claim 3, wherein the first sealant component is a low molecular weight monomer or oligomer, and the second sealant component is a catalyst or reactant.

7. The method of claim 3, wherein the first sealant component is a solution containing fibrinogen and factor XIII, and the second sealant component is a solution containing thrombin and CaCl₂.

8. The method of claim 3, wherein the first sealant contains gelatin and resorcinol, and the second sealant contains formaldehyde.

9. The method of claim 2, wherein the medical sealant material comprises one of a cyanoacrylate ester, a fibrin sealant, a gelatin-resorcinol-formaldehyde glue, an alginate and a hydrogel sealant.

10. A method of treating a heart condition comprising dispensing first and second sealant components to a myocardial infarction via an introducer extending through a percutaneous puncture in a subxiphoid region, the first and second sealant components combining to form a medical sealant material that bonds to the myocardial infarction to form a reinforcement for the myocardial infarction, wherein the reinforcement does not include a patch.

11. The method of claim 10 wherein the first sealant is dispensed first and the second sealant is dispensed onto the first sealant.

12. A myocardial infarction reinforcement consisting of a medical sealant material configured to extend over and bond to a myocardial infarction to form the reinforcement.

13. The reinforcement of claim 12, wherein the medical sealant is formed from a first sealant component combined with a second sealant component.

14. The reinforcement of claim 13, wherein the first sealant component is a low molecular weight monomer or oligomer, and the second sealant component is a catalyst or reactant.

15. The reinforcement of claim 13, wherein the first sealant component is a solution containing fibrinogen and factor XIII, and the second sealant component is a solution containing thrombin and CaCl₂.

16. The reinforcement of claim 13, wherein the first sealant contains gelatin and resorcinol, and the second sealant contains formaldehyde.

17. The reinforcement of claim 12, wherein the medical sealant material comprises one of a cyanoacrylate ester, a fibrin sealant, a gelatin-resorcinol-formaldehyde glue, an alginate and a hydrogel sealant.
